(19) United States
(12) Patent Application Publication
(10) Pub. No.: US 2022/0332689 A1
(43) Pub. Date: Oct. 20, 2022

(54) IDENTIFICATION OF COMPOUNDS THAT INHIBIT STRESS GRANULE FORMATION AND TAU AGGREGATION BY TARGETING TIAI

(71) Applicant: The Trustees Of Columbia University In The City Of New York, New York, NY (US)

(72) Inventors: Joseph B. RAYMAN, New York, NY (US); Donald LANDRY, New York, NY (US); Shixian DENG, New York, NY (US)

(21) Appl. No.: 17/760,621

(22) PCT Filed: Sep. 16, 2020
(86) PCT No.: PCT/US2020/051107

§ 371 Date: Mar. 15, 2022

Related U.S. Application Data
(60) Provisional application No. 62/900,784, filed on Sep. 16, 2019.

(51) Int. Cl. C07D 275/04; C07D 513/04; A61K 31/5377; A61K 31/428; A61K 31/437; A61K 31/519; A61K 31/506; A61K 31/4412; A61K 31/69

(52) U.S. Cl. CPC C07D 275/04; C07D 513/04; A61K 31/5377; A61K 31/428; A61K 31/437; A61K 31/519; A61K 31/506; A61K 31/4412; A61K 31/69

(57) ABSTRACT

This invention provides a method of ameliorating the symptoms of, or treating a neurodegenerative disorder, Welander distal myopathy, psychiatric illness, or cancer in a mammal, the method comprising administering to the mammal an effective amount of a compound that decreases TIA1-dependent stress granule formation.

This application claims priority of U.S. Provisional Patent Application No. 62/900,784, filed Sep. 16, 2019, the entire contents of which are hereby incorporated herein by reference.

Throughout this application, various publications are referenced by author and publication date within parentheses. Full citations for these publications may be found at the end of the specification or at the end of each experimental section. The disclosures of these publications are hereby incorporated by reference into this application to describe more fully the art to which this invention pertains.

### BACKGROUND OF THE INVENTION

TIA1 is an RNA binding protein that plays a critical role in the cellular stress response. When cells are exposed to various environmental insults (e.g., oxidative stress, viral infection, etc.), TIA1 promotes the assembly of stress granules (SGs), which are cytoplasmic foci that act as temporary storage repositories for RNAs that are not required during the stress response. These RNAs are maintained in a translationally arrested state when localized to SGs, but can be returned to the polysome pool upon resolution of environmental challenge and disassembly of SGs. In this manner, SGs facilitate preferential translation of critical RNAs during stress, and also reduce the need for de novo transcription during resumption of normal cellular functions. In addition, SGs recruit a number of key RNA binding proteins that normally serve functions in the nucleus, such as splicing factors. By altering the stoichiometry of key splicing factors, SGs modulate global alternative splicing patterns on a global scale. Finally, recruitment of signaling proteins into SGs alters cellular signaling, in particular apoptosis and cell survival. Taken together, SGs facilitate adaptive reprogramming of the proteome during cellular stress.

The functional activity of SG components such as TIA1 is partly derived from an inherent propensity to form aggregated structures under physiological conditions, which in the case of TIA1 is mediated in part by its C-terminal prion-related domain. Inventors have also recently established that multimerization of TIA1 and its recruitment into SGs is triggered by stress-dependent release of intracellular zinc, which acts as a physiological second messenger to promote reversible phase separation of TIA1 to drive SG formation (Rayman et al., 2018).

Phase separation is a general biophysical mechanism that gives rise to membrane-less organelles under appropriate physiological conditions. However, the propensity for TIA1 and other SG component proteins to undergo this type of physiological aggregation can be co-opted by a number of pathophysiological processes. Thus, it has been hypothesized that SGs can evolve into, or promote the seeding of, persistent aggregates that are cytotoxic. For example, TIA1 and SGs have been implicated in several neurodegenerative conditions, including amyotrophic lateral sclerosis (ALS), frontotemporal dementia (FTD), Alzheimer's disease (AD), and tauopathies (Maziuk et al., 2017; Mackenzie et al., 2017; Vanderweyde et al., 2016; Vanderweyde et al., 2012). In addition, mutations in TIA1 itself are associated with Welandar distal myopathy (WDM) (Hackman et al., 2013; Klar et al., 2013). A hallmark of these disorders is the presence of pathological protein inclusions that are often positive for SG components such as TIA1, TDP-43, and Tau. Together, these results and those of other studies suggest that inhibition of TIA1 activity and/or SG formation may represent a useful therapeutic approach for treating tauopathies and other neurodegenerative conditions associated with persistent protein aggregation (Fang et al., 2019; Apicco et al., 2018; Berger et al., 2007; Cowan et al., 2013; Jouanne et al., 2017; Jiang et al., 2019; Fernandes et al., 2018; Brettschneider et al., 2014; Hergesheimer et al., 2019; Neumann et al., 2006; Protter & Parker, 2016; Wolozin & Ivanov, 2019).

The functional connection between TIA1 and neurodegeneration is thought to arise from its tendency to form protein aggregates that may interact with other aggregation-prone proteins involved in these neurodegenerative processes. With respect to tau-mediated neurodegeneration, which is particularly relevant to AD and FTD, a number of compelling studies have linked TIA1, SG formation, and tau pathology. For example, interaction of TIA1 and tau regulates tau pathophysiology by modulating the generation of toxic tau oligomers (Vanderweyde et al., 2016; Jiang et al., 2019). Importantly, the cognitive deficits associated with overexpression of humanized tau in mice are reversed when the mice are crossed into a TIAL-deficient background (Apicco et al., 2018). Furthermore, kinase inhibitors that act upstream of TIAL-dependent SG formation ameliorate neurodegeneration (Vanderweyde et al., 2016). Similarly, TIA1 functionally interacts with TDP-43, a key splicing factor that becomes pathologically localized to SG-like aggregates in ALS/FTD, while pharmacological manipulations that impair TDP-43+SG formation may ameliorate cellular pathological changes (Fang et al., 2019).

### SUMMARY OF THE INVENTION

This invention provides a method of ameliorating the symptoms of, or treating a neurodegenerative disorder, Welander distal myopathy, psychiatric illness, or cancer in a mammal, the method comprising administering to the mammal an effective amount of a compound that decreases TIA1-dependent stress granule formation.

This invention also provides the compounds of any one of

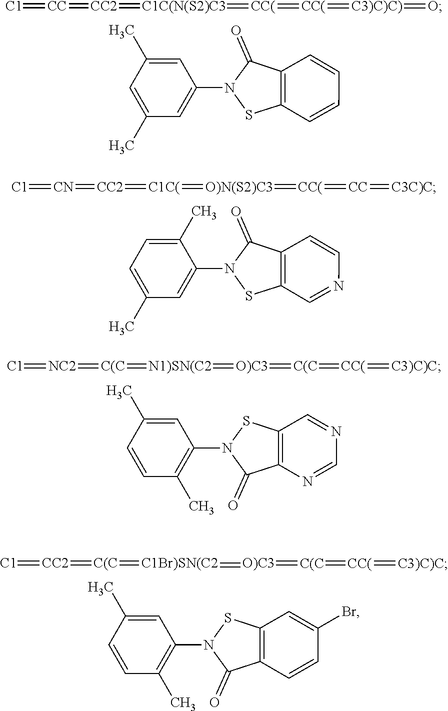

a structural analog thereof, or a pharmaceutically acceptable salt thereof.

### BRIEF DESCRIPTION OF THE DRAWINGS

FIGS. 1A-1B shows a dose-response of TIA1 compounds on arsenite-induced stress granule formation. HT22 cells (mouse brain-derived cell line) were treated with sodium arsenite (0.5 mM)+indicated compound concurrently for 30 min., fixed, and stained for endogenous TIA1. The extent of stress granule (SG) formation was determined by visual scoring of >100 cells per condition, comparing treated vs. control cells (arsenite only, no drug). FIG. 1A: Qualitative assessment of SG formation was scored on a scale of 0-3, where 0=complete inhibition of SGs and 3=same level of SG formation as in control cells treated with arsenite/vehicle. Data are rank-sorted from lowest (top row) to highest (bottom row) inhibitory potency. FIG. 1B: Representative confocal images are shown (scale bars, 10 mm).

FIGS. 2A-2B shows correlation of cellular assay (stress granule formation) with in vitro FRET data. FIG. 2A: Compounds were placed into four (4) groups based on performance in the stress granule assay. FIG. 2B: In vitro FRET data (maximum % change from baseline FRET) were averaged for each group. Overall, there is a significant statistical difference between groups. However, no correlation is detected between the magnitude of SG inhibition and FRET change for the compounds that have at least a minimal effect on SG formation (groups B, C, D).

FIG. 3 shows selected compounds sorted from least to most potent SG inhibitors, with compounds having similar potency placed within each column.

### DETAILED DESCRIPTION OF THE INVENTION

### Embodiments of the Invention

This invention provides a method of ameliorating the symptoms of, or treating a neurodegenerative disorder, Welander distal myopathy, psychiatric illness, or cancer in a mammal, the method comprising administering to the mammal an effective amount of a compound that decreases TIA1-dependent stress granule formation.

In some embodiments, the compound is any one of:

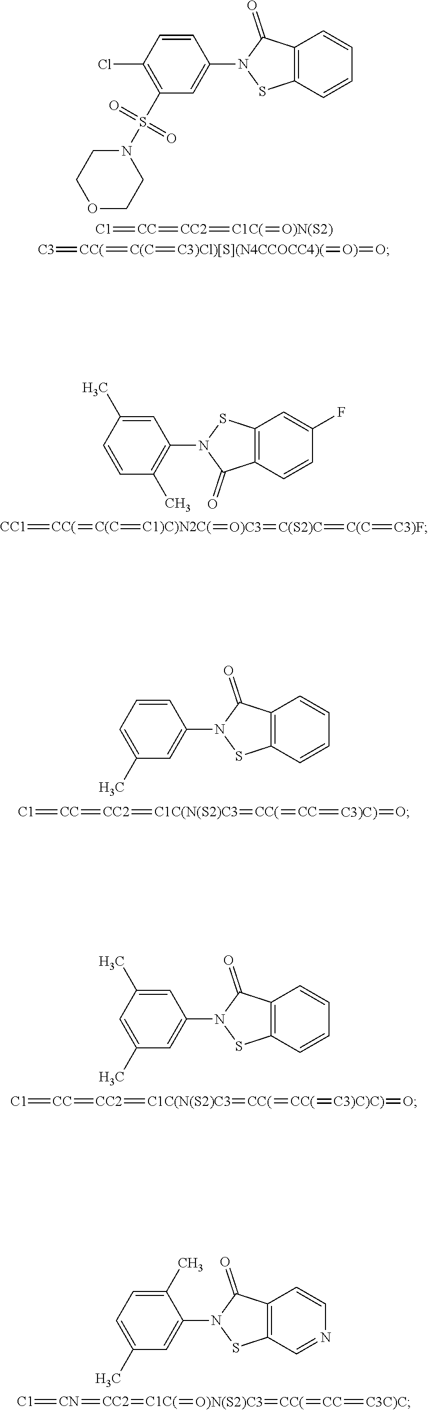

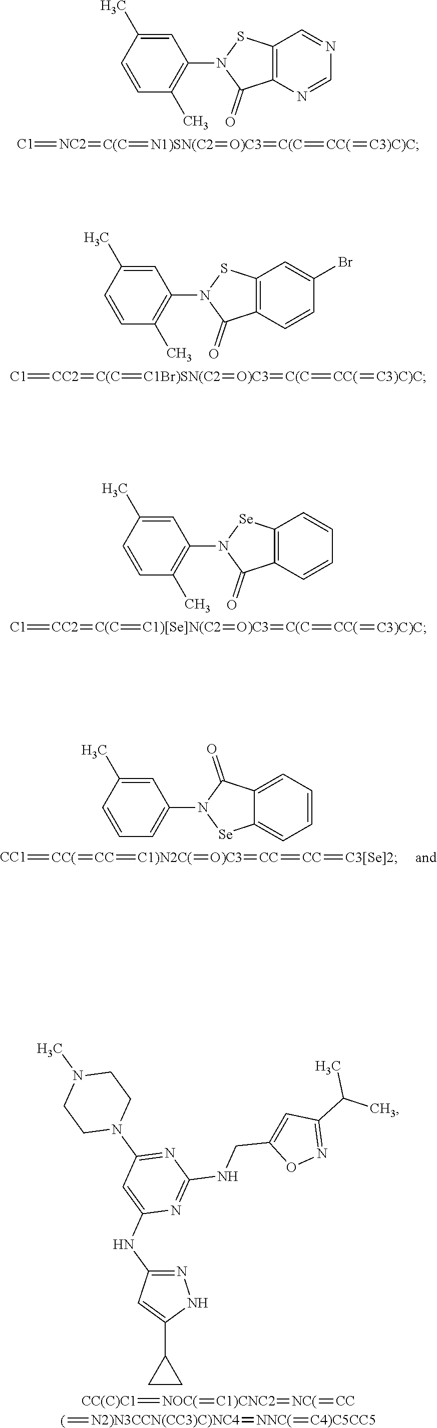

or a structural analog thereof.

In some embodiments, the compound is a pharmaceutically acceptable salt.

In some embodiments, the compound is administered in an effective amount to inhibit TIA1 multimerization.

In some embodiments, the compound is administered in an effective amount to decrease TIA1, Tau, or TDP-43 protein aggregation.

In some embodiments, the neurodegenerative disorder is any one of amyotrophic lateral sclerosis (ALS), frontotemporal dementia (FTD), Alzheimer's disease (AD), and tauopathies.

In some embodiments, the psychiatric illness is post-traumatic stress disorder (PTSD) or anxiety.

In some embodiments, the cancer is associated with a KRAS mutation.

In some embodiments, the mammal is further administered chemotherapeutic drugs.

In some embodiments, the chemotherapeutic drug is sorafenib or bortezomib.

In some embodiments, the compound is delivered to a neuron.

This invention provides compounds of any one of

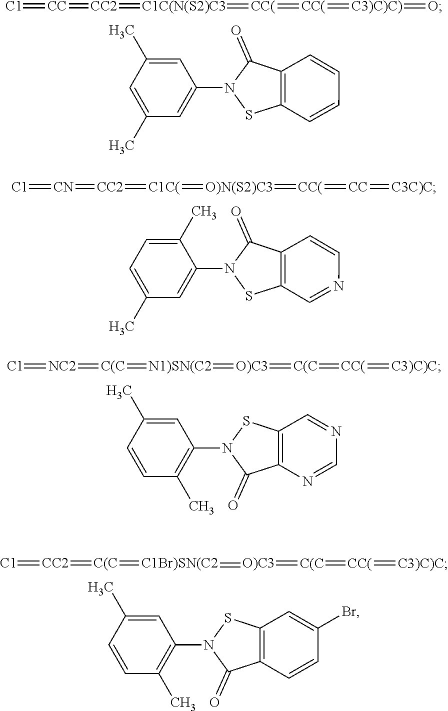

a structural analog thereof, or a pharmaceutically acceptable salt thereof.

### EXAMPLES

TIA1 is a prion-related RNA-binding protein that is strongly implicated in neurodegenerative disease, in part because of its ability to promote aggregation of disease-associated proteins. The inventors of this disclosure have identified several compounds that target the ability of TIA1 to form macromolecular aggregates in both in vitro and in cell culture models. Although inhibition of signaling events upstream of TIA1 activity has been shown to block neurodegeneration in animal studies, such strategies are associated with significant toxicity and non-specific effects (Maziuk et al., 2017). In contrast, the compounds identified herein are more specific to TIA1, and show minimal toxicity in animal studies. Accordingly, these compounds are invaluable for use as therapeutics and in the development of additional novel therapeutics that inhibit a variety of neurodegenerative processes in humans, which have become increasingly common among the aging population and represent a major health concern.

More specifically, compounds that target TIA1 represent a novel class of drugs that are of therapeutic utility in treating neurodegenerative disorders including, but not limited to, Alzheimer's disease (AD), frontotemporal lobar degeneration (FTLD), amyotrophic lateral sclerosis (ALS), etc., each of which currently has no treatment options offering effective and long-lasting amelioration.

This disclosure identifies TIA1 as a novel therapeutic target and shows that compounds that target TIA1 represent a novel class of compounds with mechanisms of action that are completely distinct from those of currently available drugs. As opposed to technologies that target tau itself, upstream enzymes that act on tau, microtubules, and so on, TIA1 is a compelling therapeutic target because of its proximity to SG formation. Indeed, manipulations that target SG formation upstream of TIA1 (e.g., PERK inhibitors, eIF2-alpha phosphorylation inhibitors, etc.) produce more toxic, non-specific effects than targeting a proximal component like TIA1. Also, given that TIA1 deletion in mice is associated with relatively mild phenotypic changes, it is a more effective therapeutic target than a gene product whose deletion is associated with lethality (for example, TDP-43). Accordingly, the compounds described herein display less toxicity and non-specific effects than other compounds that target the TIA1/tau/stress granule pathway.

Additional applications for the compounds described herein include, but are not limited to, treatment of Welander distal myopathy, a rare disorder caused by a missense mutation in the human TIA1 gene. Furthermore, these compounds may be used to modulate fear memory, which is relevant to psychiatric illnesses such as PTSD and anxiety. In addition, these compounds may be relevant to oncological indications. For example, several types of cancers, such as those with KRAS mutations, form SGs in response to chemotherapeutic agents to mitigate their cytotoxic effects. Inventors have shown that TIA1 antagonists block the formation of SGs in selected cancer cell lines treated with chemotherapeutic drugs such as sorafenib or bortezomib. These results suggest that inhibition of SG formation in cancer cells may render them more susceptible to the cytotoxicity of chemotherapeutic drugs. Additionally, TIA1 antagonists have potential therapeutic utility for use as adjuvants to existing chemotherapeutic approaches.

In Vitro and Cell-Based Studies

Based on a high-throughput drug screen that the inventors have developed (as described in Rayman et al., 2018), they initially identified several commercially available compounds that target multimerization of TIA1 and its ability to promote SG formation. In cell culture experiments using both human and mouse cell lines, at least two of these compounds (described below) completely blocked 1) TIA1-dependent SG formation, 2) recruitment of tau/TDP-43 into SGs, and 3) colocalization of TIA1, tau, and TDP-43. These effects were observed with as little as 10 μM drug in several different cell lines. In a typical assay, SGs are induced by treating cells (e.g., HT22 or SH-SY5Y cell lines) with sodium arsenite (0.5 mM) for 30 min., followed by fixation and immunocytochemical analysis. Drugs were administered concurrently with sodium arsenite. Fixed cells were then stained for endogenous TIA1 followed by confocal imaging and analysis. Inventors have also established that the active compounds also block puromycin-induced SGs in human motor neurons, which represents a more translationally relevant system for studying ALS. Interestingly, IC50 values for the active compounds are 5-10× lower in primary neurons compared to cell lines, although this finding may be explained in part by the use of different stressors (e.g. arsenite for cell lines and puromycin for motor neurons). Inventors also demonstrate that these compounds can accelerate the disassembly of pre-formed SGs in various experimental contexts.

Furthermore, inventors have established that the active compounds effectively block SG formation involving disease-related SG components. For example, in transfection studies, the compounds fully prevent SG assembly by WDM-TIA1, which bears the causative mutation for Welander distal myopathy, and is associated with abnormally persistent SGs in human cells. In addition, the active compounds efficiently block puromycin-induced SG assembly in human motor neurons harboring mutations in defined ALS susceptibility loci (for example, in motor neurons with C9orf72, SOD1, or FUS mutations). Interestingly, inventors also observed that aberrantly persistent SGs formed in human FUS mutant neurons can also be disassembled by our small molecules. Together, these results suggest that the compounds disclosed herein can universally target SG formation and disassembly across a range of distinct experimental contexts.

Thus far, a cluster of available structural analogs have been tested. More recently, the inventors have also developed and tested several novel chemical entities based on the structure of the earlier compounds. Additional novel analogs may be synthesized based on identified structure-activity relationships.

Active compounds in simplified molecular-input line-entry system (SMILES) format
Ref 35: C1=CC=CC2=C1C(=O)N(S2)C3=CC(=C(C=C3)C1) [S](N4CCOCC4)(=O)=O
Ref 49: CC1=CC(=C(C=C1)C)N2C(=O)C3=C(S2)C=C(C=C3)F
OTX1000: C1=CC=CC2=C1C(N(S2)C3=CC(=CC=C3)C)=O
OTX1008: C1=CC=CC2=C1C(N(S2)C3=CC(=CC(=C3)C)C)=O
OTX1013: C1=CN=CC2=C1C(=O)N(S2)C3=CC(=CC=C3C)C
OTX1014: C1=NC2=C(C=N1)SN(C2=O)C3=C(C=CC(=C3)C)C
OTX1015: C1=CC2=C(C=C1Br)SN(C2=0)C3=C(C=CC(=C3)C)C
OTX1016: C1=CC2=C(C=C1) [Se]N(C2=0)C3=C(C=CC(=C3)C)C
OTX1019: CC1=CC(=CC=C1)N2C(=O)C3=CC=CC=C3[Se]2
OTX00E9:
CC(C)C1=NOC(=C1)CNC2=NC(=CC(=N2)N3CCN(CC3)C)NC4=NNC(=C4)C5CC5

Dose-Response of TIA1 Compounds on Arsenite-Induced Stress Granule Formation

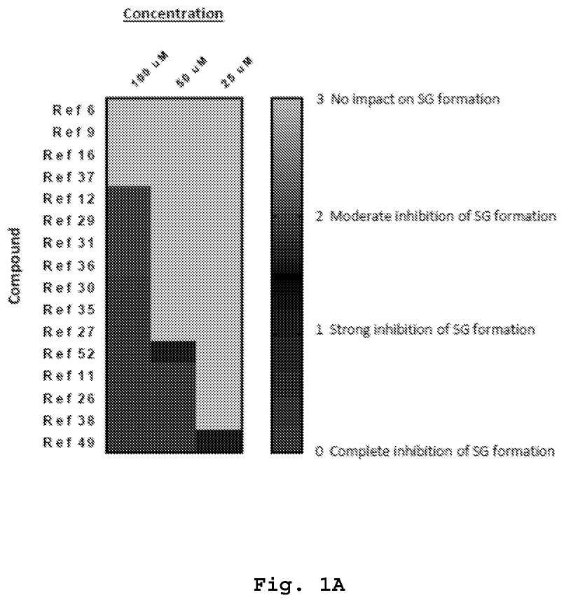

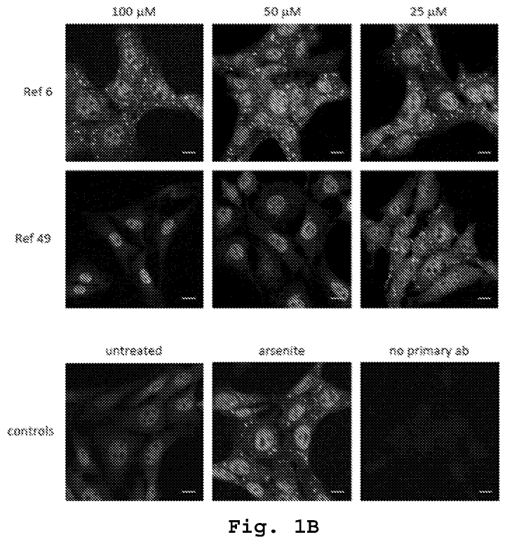

Protocol: HT22 cells (mouse brain-derived cell line) were treated with sodium arsenite (0.5 mM)+indicated compound concurrently for 30 min., fixed, and stained for endogenous TIA1. The extent of stress granule (SG) formation was determined by visual scoring of >100 cells per condition, comparing treated vs. control cells (arsenite only, no drug). Qualitative assessment of SG formation was scored on a scale of 0-3, where 0=complete inhibition of SGs and 3=same level of SG formation as in control cells treated with arsenite/vehicle (FIG. 1A). Representative confocal images are shown (scale bars, 10 mm) (FIG. 1B). Data are rank-sorted from lowest (top row) to highest (bottom row) inhibitory potency (FIG. 1A). All compounds except for Ref 16 were positive in the in vitro FRET assay described in PCT International Publication No. WO/2017/218697.

Correlation of Cellular Assay (Stress Granule Formation) with In Vitro FRET Data

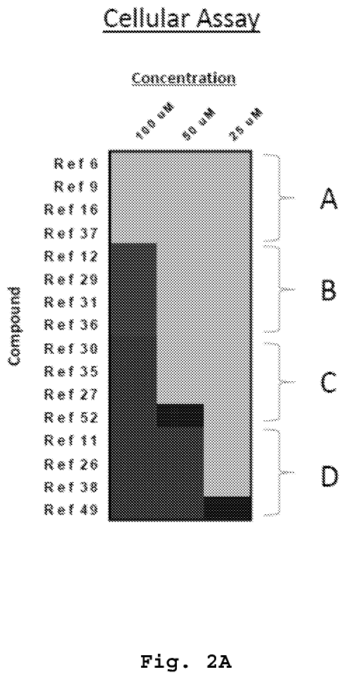

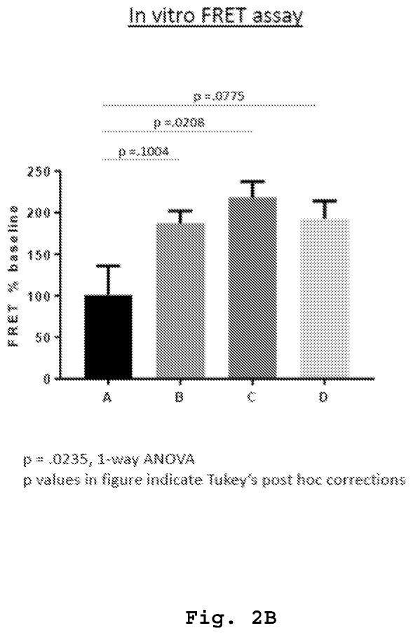

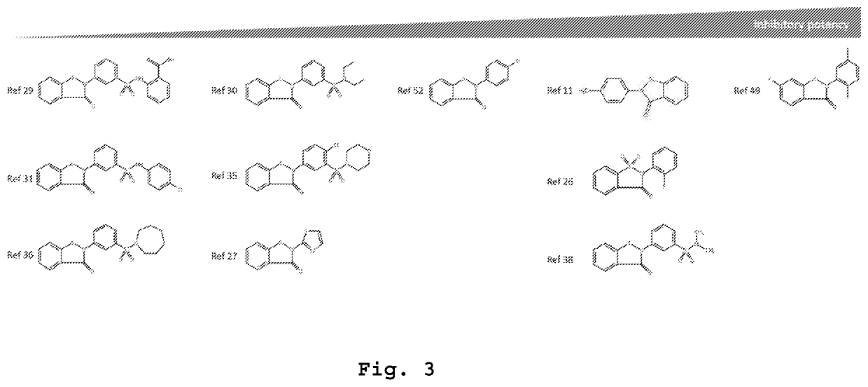

Compounds were placed into four (4) groups based on performance in the stress granule assay (FIG. 2A). In vitro FRET data (maximum % change from baseline FRET) were averaged for each group (FIG. 2B). Overall, there is a significant statistical difference between groups. However, no correlation is detected between the magnitude of SG inhibition and FRET change for the compounds that have at least a minimal effect on SG formation (groups B, C, D).

### REFERENCES

Apicco et al., “Reducing the RNA binding protein TIA1 protects against tau-mediated neurodegeneration in vivo” (2018), Nat Neurosci. 21(1):72-80
Berger et al., “Accumulation of pathological tau species and memory loss in a conditional model of tauopathy” (2007), J Neurosci., 27(14):3650-62
Brettschneider et al., “TDP-43 pathology and neuronal loss in amyotrophic lateral sclerosis spinal cord” (2014) Acta Neuropathol. 128(3):423-37
Cowan et al., “Are tau aggregates toxic or protective in tauopathies?” (2013), Front Neurol. 4:114
Fang et al., “Small-Molecule Modulation of TDP-43 Recruitment to Stress Granules Prevents Persistent TDP-43 Accumulation in ALS/FTD” (2019), Neuron., 103(5):802-819
Fernandes et al., “Stress Granules and ALS: A Case of Causation or Correlation?” (2018), Adv Neurobiol. 20:173-212
Hackman et al., “Welander distal myopathy is caused by a mutation in the RNA-binding protein TIAL” (2013), Ann Neurol., 73(4):500-9
Hergesheimer et al., “The debated toxic role of aggregated TDP-43 in amyotrophic lateral sclerosis: a resolution in sight?” (2019) Brain 142(5):1176-1194
Jiang et al., “TIA1 regulates the generation and response to toxic tau oligomers” (2019), Acta Neuropathol. 137(2):259-277
Jouanne et al., “Tau protein aggregation in Alzheimer's disease: An attractive target for the development of novel therapeutic agents” (2017), Eur J Med Chem. 139:153-167
Klar et al., “Welander distal myopathy caused by an ancient founder mutation in TIA1 associated with perturbed splicing” (2013), Hum Mutat., 34(4):572-7
Mackenzie et al., “TIA1 Mutations in Amyotrophic Lateral Sclerosis and Frontotemporal Dementia Promote Phase Separation and Alter Stress Granule Dynamics” (2017), Neuron, 95(4):808-816
Maziuk et al., “Dysregulation of RNA Binding Protein Aggregation in Neurodegenerative Disorders” (2017), Front Mol Neurosci; 10:89
Neumann et al., “Ubiquitinated TDP-43 in frontotemporal lobar degeneration and amyotrophic lateral sclerosis” (2006) Science 314:130-133
Protter & Parker, “Principles and Properties of Stress Granules” (2016) Trends Cell Biol. 26(9):668-679
Rayman et al., “TIA-1 Self-Multimerization, Phase Separation, and Recruitment into Stress Granules Are Dynamically Regulated by Zn2” (2018), Cell Rep., 22(1):59-71
Vanderweyde et al., “Contrasting pathology of the stress granule proteins TIA-1 and G3BP in tauopathies” (2012), J Neurosci., 32(24):8270-83
Vanderweyde et al., “Interaction of tau with the RNA-Binding Protein TIA1 Regulates tau Pathophysiology and Toxicity” (2016), Cell Rep., 15(7):1455-1466
Wolozin & Ivanov, “Stress granules and neurodegeneration” (2019) Nat Rev Neurosci. 20(11):649-666

1. A method of ameliorating the symptoms of, or treating a neurodegenerative disorder, Welander distal myopathy, psychiatric illness, or cancer in a mammal, the method comprising administering to the mammal an effective amount of a compound that decreases TIA1-dependent stress granule formation.

2. The method of claim 1, wherein the compound is any one of: 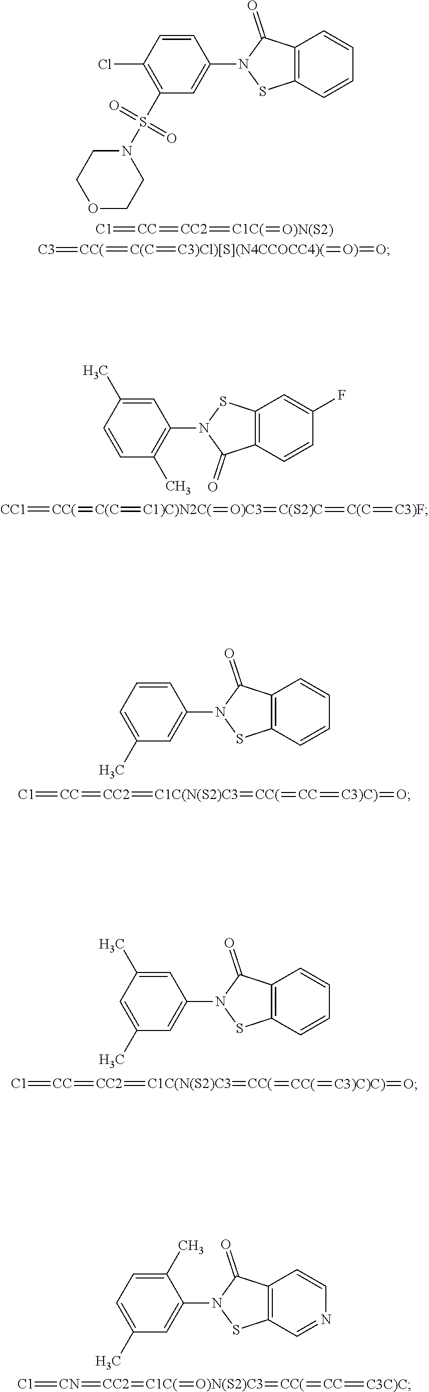 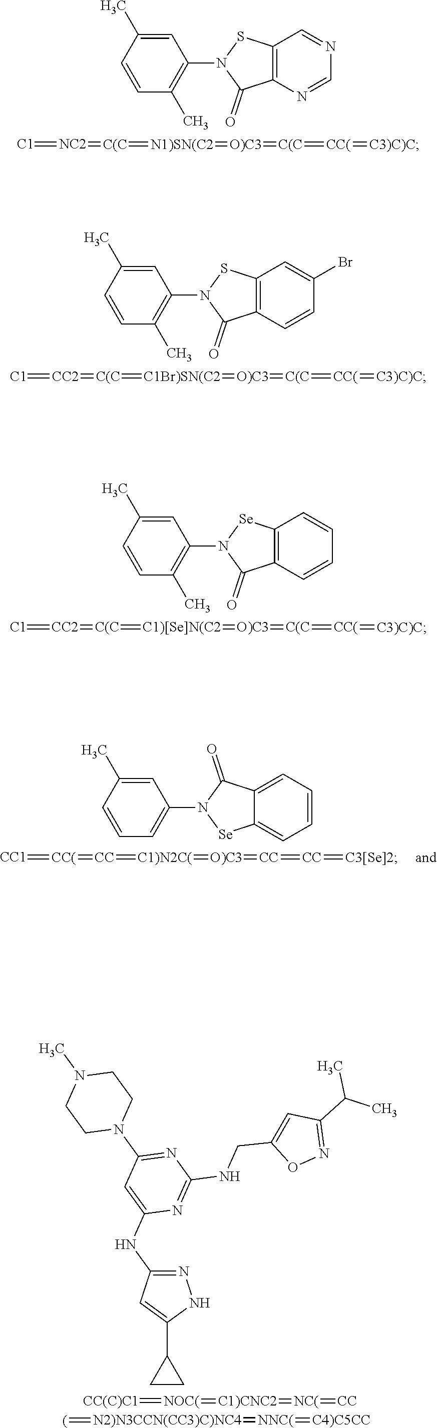
or a structural analog thereof.

3. The method of claim 1, wherein the compound is a pharmaceutically acceptable salt.

4. The method of claim 1, wherein the compound is administered in an effective amount to inhibit TIA1 multimerization.

5. The method of claim 1, wherein the compound is administered in an effective amount to decrease TIA1, Tau, or TDP-43 protein aggregation.

6. The method of claim 1, wherein the neurodegenerative disorder is any one of amyotrophic lateral sclerosis (ALS), frontotemporal dementia (FTD), Alzheimer's disease (AD), and tauopathies.

7. The method of claim 1, wherein the psychiatric illness is post-traumatic stress disorder (PTSD) or anxiety.

8. The method of claim 1, wherein the cancer is associated with a KRAS mutation.

9. The method of claim 1, wherein the mammal is further administered chemotherapeutic drugs.

10. The method of claim 9, wherein the chemotherapeutic drug is sorafenib or bortezomib.

11. The method of claim 1, wherein the compound is delivered to a neuron.

12. A compound comprising any one of the following: 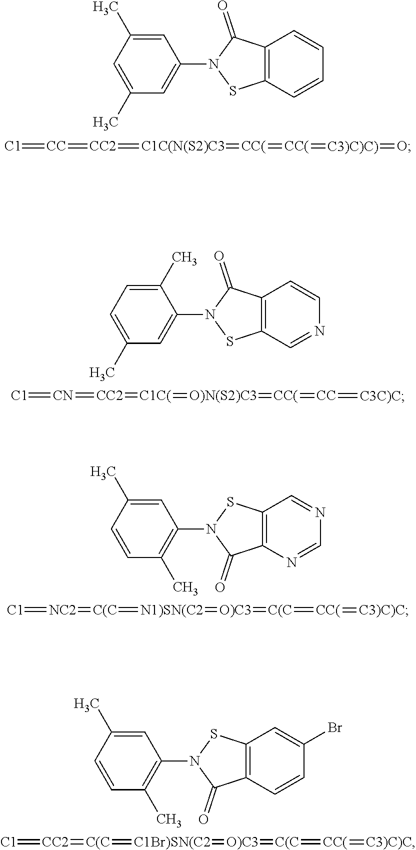
a structural analog thereof, or a pharmaceutically acceptable salt thereof.
